(19) United States
(12) Patent Application Publication
(10) Pub. No.: US 2012/0053130 A1
(43) Pub. Date: Mar. 1, 2012

(54) COMPOSITION FOR ENHANCING ABSORPTION OF A DRUG AND METHOD

(75) Inventors: Neil R. Mathias, North Brunswick, NJ (US); Lianli Li, Pomona, NY (US)

(73) Assignee: Bristol-Myers Squibb Company

(21) Appl. No.: 12/871,460

(22) Filed: Aug. 30, 2010

(51) Int. Cl. A61K 38/12; A61K 31/522; A61K 31/165

(52) U.S. Cl. 514/21.1; 514/620; 514/263.37

(57) ABSTRACT

A composition for enhancing absorption of a pharmaceutical which may have poor oral bioavailability, which composition has surprisingly little cytotoxicity, is provided which is in the form of a liquid or semi-solid or solid containing an admixture (1) a mucoadhesive polymer which is a polyacrylic acid polymer, preferably Carbopol 971P, and (2) an absorption or permeation enhancer which preferably is L-α-lyso-phosphatidylcholine (LPC), and which composition is free of polysaccharides. A method for improving bioavailability of a drug which has poor absorption properties is also provided wherein the above bioadhesive composition is administered with said pharmaceutical to the mucosal membrane of the GI tract, nose, oral cavity, sublingual, buccal, and vaginal mucosa.

A method for reducing the cytotoxic effect of an absorption enhancer such as LPC is also provided wherein a mucoadhesive polymer as described above is administered with the LPC to a patient in need of treatment.

This application claims a benefit of priority from U.S. Provisional Application No. 60/566,049, filed Apr. 28, 2004, the entire disclosure of which is herein incorporated by reference.

### FIELD OF THE INVENTION

The present invention relates to a composition which enhances bioavailability of therapeutic agents which may be poorly absorbed, which composition contains a mucoadhesive and an absorption enhancer, and has surprisingly reduced toxicity as compared to previously known absorption enhancing compositions, to a method for improving bioavailability of poorly absorbable therapeutic agents via oral or topical delivery to mucosal membranes employing such composition, and to a method for reducing cytotoxic effects of an absorption enhancer (employed to improve bioavailability of poorly absorbed therapeutic agents) thereby providing more tolerable delivery to mucosal membranes, employing a special mucoadhesive in combination with the absorption enhancer.

### BACKGROUND OF THE INVENTION

Therapeutic peptide and protein macromolecules and poorly permeable small molecule pharmaceutical agents are often poorly absorbed through oral and other mucosa due to the limitations of their physicochemical properties (size, charge, solubility), poor epithelial permeability, or susceptibility to metabolizing enzymes. Permeation enhancers such as surfactants, fatty acids, fatty alcohols, bile salts and bile acids, sugar esters and chelators have been employed to increase the bioavailability and extent of absorption of such compounds. The permeation enhancers increase the permeability of a mucosal barrier and facilitate the diffusion of an active drug across the mucosal barrier by disrupting the mucosal barrier either by opening tight junctions between adjacent epithelial cells (paracellular pathway) or by fluidizing phospholipid membranes to allow better diffusion of the active drug across the bilayer (transcellular pathway). Aungst, B. J., 2000, “Intestinal permeation enhancers”, J. Pharm. Sci. 89:429-42; Hochman, T. et al., “Mechanisms of absorption enhancement and tight junction regulation”, J. Control. Rel. 29:253-267. However, many of these agents are toxic or irritating to mucosal surfaces. The high local concentration of permeation enhancer at the site of administration causes a sustained enhancing effect with poor reversibility. Thus, the tissue is vulnerable to adverse inflammatory response, which raises safety and tolerability concerns for practical chronic or acute use of formulations containing such enhancers. Accordingly, there is, indeed, a long felt need in the pharmaceutical industry for a formulation which enhances bioavailability of therapeutic agents without causing toxic or irritating effects on mucosal surfaces.

Combining mucoadhesive polymers with a permeation enhancer can help retain the pharmaceutical drug and enhancer at the site of absorption. Mucoadhesion is a characteristic of natural or synthetic polymers that bind to the mucous lining creating intimate contact with the absorptive mucosal membranes. The high concentration of carboxylate groups of the polymer form H-bonds and ionic interactions with the mucosal surface, while the long intertwined polymer backbone entrap components of the composition. This results in improved absorption of a poorly absorbed pharmaceutical agent over a prolonged duration.

Mucoadhesives commonly used in pharmaceutical preparations include: carboxymethylcellulose (CMC), hydroxypropylmethylcellulose (HPMC), polyacrylic and polymethacrylic acid and their derivatives, pectin, alginic acid, chitosan, polyvinylpyrrolidone, hyaluronic acid, and polyvinyl alcohol.

Carbopol® polymers (Noveon, Inc.) are cross-linked polyacrylic acids commonly used as a pharmaceutical excipient in oral suspensions, tablets and sustained release formulations.

L-α-Lysophosphatidylcholine (LPC), a natural metabolite of phosphatidylcholine in biological membranes, has been demonstrated to be a potent enhancer of peptide absorption (e.g., Fisher, A. N. et al., “Effect of L-alpha-lysophosphatidylcholine on the nasal absorption of human growth hormone in three animal species”, Int. J. Pharm., 1991, 71:147-156). However, when applied on epithelia as an absorption enhancer, LPC has been shown to cause significant disturbance of membrane fluidity and thus cell damage (Chandler, S. G. et al., “Nasal absorption in rats. II. Effect of enhancers on insulin absorption and nasal histology”, hit. J. Pharm., 1991, 76:61-70; Richardson, J. L. et al., “Vaginal absorption of insulin in the rat: effect of penetration enhancers on insulin uptake and mucosal histology”, Pharm. Res., 1992, 9:878-883; Martlin, E. et al., “Effect of absorption enhancers on rat nasal epithelium in vivo: release of marker compounds in the nasal cavity”, Pharm. Res., 1995, 8:1151-1157), raising concerns about the safety of using LPC as an absorption enhancer.

The patent literature is replete with examples of permeation enhancers which effectively increase permeability of drugs. Many of these references involve transdermal delivery and irritation reduction in skin for transdermal delivery. Skin is the most robust and resilient bather in the body and offers little resemblance to mucosal barriers of the gastro-intestinal, nasal, buccal, sublingual, ocular and vaginal cavity. As indicated below, some patents disclose compositions that contain surfactants for the purpose of wetting, solubilization or composition stabilization in amounts and concentrations different from those used for drug permeation enhancement. It is also known to include polymers as gelling agents to provide body to the formulation.

U.S. Pat. No. 5,744,155 to Friedman et al. discloses oil-in-water emulsions which include a collodial dispersion of droplets or particles having a hydrophobic core and containing a mucoadhesive such as an acrylic acid polymer, and may include a surfactant such as a phospholipid such as a phosphatidylcholine and a cosurfactant which may be lysophosphatidylcholine to stabilize the emulsion.

U.S. Pat. No. 6,319,913 to Mak et al. discloses a penetration enhancing gel composition which enhances penetration of transdermally or topically applied drugs while providing reduced skin irritation compared to that which often accompanies transdermal or topical drug delivery systems. The penetration enhancing gel is formed of oleic acid, ethanol, propylene glycol, a gelling agent (Carbopol), additional irritation reducing agents and drug to be delivered such as testosterone. Mak et al. disclose that the membrane fluidizing enhancer oleic acid was less irritating than oleyl alcohol in a formulation containing a C₁-C₄ alcohol cosolvent and a gelling agent like a polyacrylic acid polymer to retain drug on the skin.

U.S. Patent Application Publication No. U.S. 2003/0143277 A1 to Ameye et al. filed on Jan. 31, 2002, published Jul. 31, 2003, discloses a solid bioadhesive composition in the form of a tablet or powder, which may include a therapeutic agent, having improved bioadhesive properties over prior art bioadhesive compositions, for example, improved mucoadhesive properties which results in increased drug loading capacity and a reduced incidence of mucosal irritation. In one aspect, the bioadhesive composition is formed of an intimate mixture of (1) a polysaccharide, such as a waxy starch, and (2) a polycarboxylated polymer such as Carbopol polymers. The bioadhesive composition may also include (3) an absorption enhancer such as a synthetic surfactant, non-ionic surfactant, steroidal surfactant, bile salt, chelator, fatty acid or derivative thereof, sugar ester such as phosphatidylcholine (present from 0.001 to 10% by weight). The intimate mixture is prepared in such a way that each particle is formed of a mixture of polysaccharide and polycarboxylated polymer as opposed to discrete particles of polysaccharide and polycarboxylated polymer.

U.S. Pat. No. 6,503,894 to Dudley et al. discloses a hydroalcoholic gel for percutaneous administration which gel includes 30 to 98% w/w of a lower alcohol such as ethanol or isopropanol, a penetration enhancer which is a functional derivative of a fatty acid which can be isopropyl myristate and a thickener such as a polyacrylic acid such as a Carbopol® polymer.

U.S. Pat. No. 6,309,663 to Patel et al. discloses compositions and methods to effect enhanced absorption of hydrophilic therapeutic agents. The compositions include an absorption enhancing carrier, where the carrier is formed from a combination of at least two surfactants, at least one of which is hydrophilic. A hydrophilic therapeutic agent can be incorporated into the composition or can be coadministered with the composition. The dosage form containing the combination of at least two surfactants which may include an ionizable surfactant which can be lyso-phosphatidylcholine (LPC) and a non-ionizable surfactant, can be coated with mucoadhesive polymers such as polyacrylate derivates for sustained release or enteric coating.

U.S. Pat. No. 5,631,004 to Cagle et al. discloses a composition in the form of a viscous gel or solid insert for sterilizing ocular tissue prior to surgery, which includes an antimicrobial in a viscous polymer gelling agent such as Carbopol, and an enhancer of ocular permeation which can be a saccharide such as dodecylmaltoside or a monoacyl phosphoglyceride such as lysophosphatidylcholine.

Literature references that describe the ability of formulations to reduce the toxicity of enhancers include, Velardi, A. L. M. et al., “Cell type-dependent effect of a phospholipid and cholesterol on bile salt cytotoxicity”, Gastroenterology, 1991, 101:457-464, where phosphatidylcholine protects cells from the toxic effects of bile salts in Caco-2 cells by forming mixed micelles. Werner, U. et al., “Effect of permeation enhances on the transport of a peptidomimetic thrombin inhibitor (CRC220) in a human intestinal cell line (Caco-2)”, Pharm. Res., 1996, 13:1219-1227.

Gould, L. A. et al., “Mitigation of surfactant erythrocyte toxicity by egg phosphatidylcholine”, J. Pharm. Pharamcol., 2000, 52:1203-1209, reported that surfactants affected by egg phosphatidylcholine include polyoxyethylene alkyl ethers (Brij), sodium dodecyl sulfate, lysophosphatidylcholine and tetradecyltrimethylene ammonium bromide.

While the prior art may describe specific enhancers, polymers or mixtures thereof that show lesser irritation than others, it does not teach the art of reducing irritation of strong permeation enhancers and toxic surfactants, while retaining or boosting absorption/bioavailability of an active drug.

### BRIEF DESCRIPTION OF THE INVENTION

In accordance with the present invention an absorption enhancing composition, preferably in fluid form, for enhancing absorption of pharmaceutically active drugs is provided which composition has surprisingly reduced cytotoxicity. The composition of the invention is formed of an admixture of

(a) a mucoadhesive polymer; and

(b) an absorption or permeation enhancer,

which composition is designed for delivery to mucosal membranes, excluding skin or percutaneous tissue, or systemic delivery to the blood stream, to facilitate diffusion of a poorly permeable drug, which composition is free of polysaccharides, and includes a drug which may be a poorly absorbable drug or may be employed separately, in combination, with the drug.

The composition of the invention will not include an alcohol (other than to solubilize a pharmaceutical agent), so as to avoid irritation to the mucosal membranes, or an oil-in-water emulsion which may be difficult to handle due to poor composition stability.

In a preferred embodiment of the invention, the mucoadhesive polymer is a carboxylated polymer, such as a polyacrylic acid or cross-linked polyacrylic acid, and the absorption enhancer is a lysophosphatidate, preferably lysophosphatidylcholine, more preferably L-α-lysophosphatidylcholine (LPC), also known as lysolecithin.

LPC is a natural metabolite of phosphatidylcholine. It is theorized that when LPC is applied to biological membranes, it disrupts epithelial membrane fluidity and tight junction integrity thereby facilitating the absorption of a poorly permeable pharmaceutical.

The absorption enhancer composition of the invention enhances delivery of poorly absorbable drugs to mucosal tissue, such as mucosal membranes of the gastrointestinal tract, nose, oral cavity, sublingual, buccal, rectal, uteral, bladder, pulmonary, and vaginal mucosa, excluding the mucosa of the eye. Delivery of the drug from or with the composition of the invention is accomplished by bioadhesion of the composition and drug to mucosal membranes and via the oral or topical route and does not include transdermal delivery or other delivery systems for delivering drug percutaneously, such as to skin or tissue.

Through use of the combination of the mucoadhesive polymer and absorption enhancer in accordance with the present invention, superior permeation enhancement is achieved over that afforded by the enhancer alone, while attaining reduction in cytotoxicity or irritation potential of the permeation enhancer.

In addition, in accordance with the present invention, a method is provided for improving bioavailability of a drug which may have poor absorption properties which includes the steps of administering to designated mucosal membranes of a patient in need of treatment a bioadhesive composition, preferably, in fluid form, which is formed of an admixture of

1) a mucoadhesive polymer; and

2) an absorption enhancer, which composition is free of polysaccharides and has surprisingly reduced cytotoxicity as compared to previously known absorption enhancing compositions, and which composition may optionally include a drug which may be a poorly absorbable drug or may be employed separately, in combination, with the drug.

Still further, in accordance with the present invention, a method is provided for reducing the cytotoxic effect and inflammation which may be caused by an absorption enhancer, which method includes the step of administering to a patient in need of treatment an absorption enhancer together with a mucoadhesive polymer, and a pharmaceutical, whereby permeation of said pharmaceutical into local tissue or system circulation is enhanced while normally expected cytotoxic effect of the absorption enhancer is reduced.

It has been found that the mucoadhesive polymer Carbopol 971 P has a cytoprotection effect by reducing membrane damage caused by the absorption enhancer L-α-lysophosphatidylcholine. Thus, in accordance with the present invention, a method is provided for reducing the cytotoxic effect of the permeation enhancer L-α-lysophosphatidylcholine during the administration of a pharmaceutical, which method includes the step of administering the L-α-lysophosphatidylcholine and pharmaceutical together with a polycarboxylated polymer, such as a polyacrylic acid polymer, preferably Carbopol 971P.

### BRIEF DESCRIPTION OF THE FIGURES

FIG. 1 is a graph showing effect of 0.5% Carbopol 971P and its combination with L-α-lysophosphatidylcholine on permeability of 1-diamino-8-D-arginin-vasopressin (DDAVP) across Calu-3 cells;

FIG. 2 is a graph showing an Alamar Blue Assay depicting black and gray bars wherein the black bar indicates % of cell survival after 2 hrs treatment of LPC at 0-0.25% and the gray bar indicates the % cell survival after 2 hrs treatment of the combination of LPC with 0.5% of Carbopol 971P;

FIG. 3 is a graph showing % of transepithelial electrical resistance (TEER) after 2 hours of treatment with L-α-lysophosphatidylcholine, and a combination of L-α-lysophosphatidylcholine and Carbopol 971 P;

FIG. 4 is a graph showing ability of Carbopol 971P/LPC to reduce cytotoxicity in rat colon;

FIG. 5 is a bar graph which shows the availability of Carbopol 971P/LPC to reduce cytotoxicity in rat colon as measured by lactate dehydrogenase release in rats;

FIG. 6 is a graph which shows enhancement of intra-nasal DADLE absorption in rabbits by a combination of Carbopol 971P/LPC over LPC alone; and

FIG. 7 is a bar graph which shows toxicity of various formulations administered to sensitive mucosa of the rabbit nasal cavity.

### DETAILED DESCRIPTION OF THE INVENTION

The composition of the invention provides a nontoxic and effective permeation enhancement drug delivery system. In a preferred embodiment, the composition of the invention provides a macromolecule delivery system with both cytoprotective and permeation enhancement which is formed of the mucoadhesive polymer and the absorption enhancer. In the most preferred embodiment of the invention the mucoadhesive polymer is Carbopol 971P and the absorption enhancer is a lysophosphatidate, preferably L-α-lysophosphatidylcholine (LPC). It has been found that the Carbopol 971P significantly reduces the cytotoxicity of the LPC while dramatically enhancing the transepithelial absorption of peptides such as the nonapeptide, 1 deamino-8-D-arginin-vasopressin (DDAVP).

The composition of the invention affords at least two important benefits: superior permeation enhancement than that afforded by the enhancer alone, and reduction in cytotoxicity or irritation potential of the permeation enhancer. Generally, the more potent a permeation enhancer, the greater the associated cytotoxicity. This compromises the overall utility of compositions containing such an enhancer. This invention is unique in that the composition potentiates the enhancement of the permeation enhancer while simultaneously attenuating its toxicity and irritation properties at the site of delivery, thus improving the overall safety of the composition.

The extent of permeation enhancement of an active pharmaceutical ingredient from compositions in this invention is greater than that afforded by the individual components. LPC is a potent enhancer, but when combined with polyacrylic acid mucoadhesive polymers the enhancement is often the sum of the enhancement potential of the surfactant enhancer and the polymer (additive effect) (Examples 7 and 9). It is theorized that the reason for this is probably related to complementary mechanism of epithelial barrier disruption that leads to drug transport enhancement. LPC increases transcellular diffusion by fluidizing phospholipid membranes and polyacrylic acid polymers increase paracellular diffusion by destabilizing epithelial tight junctions via Ca⁺²-chelation from proteins that make up the tight junctional complex.

Concomitant with superior enhancement, the polyacrylic acid polymer in the composition of the invention has the ability to attenuate the cellular toxicity or irritation response of the surfactant permeation enhancer at the site of administration (Examples 5, 6, 8, and 10). This effect was observed in vitro (Examples 5 and 6) and in vivo in different mucosal tissues and in different species (Examples 8 and 10) indicating the feasibility of broad application. Furthermore, the mucoadhesive polymer aids in the recovery process enabling the epithelial lining to restore its natural barrier properties post treatment with the permeation enhancer (Example 6).

Other viscosity building bioadhesive and gelling agents, such as the cellulosic polymers, hydoxypropylmethylcellulose and carboxymethylcellulose do not show the permeability enhancement advantage observed with polyacrylic acid polymers. They do not disrupt epithelial tight-junctions, and therefore, do not contribute to permeability enhancement of an active pharmaceutical ingredient (Example 12). Likewise, other types of permeation enhancers do not offer any advantage when combined with polyacrylic acids. For example, phosphatidylcholine, EDTA and sodium caprate do not show additive permeability enhancement advantage or change in epithelial resistance in the presence of Carbopol polymers (Example 11).

In carrying out the present invention, including the composition, and method for improving bioavailability of a drug and method for reducing cytotoxic effect of an absorption enhancer, the absorption enhancer will be employed in a weight ratio to the mucoadhesive polymer within the range from about 0.01:1 to about 10:1, preferably from about 0.1:1 to about 5:1.

The composition of the invention containing mucoadhesive polymer and absorption enhancer will preferably be in fluid form, that is it will be in the fowl of a solution, suspension or semi-solid, such as a gel, preferably a solution. To this end, where the composition is in the form of a solution or suspension, the composition may include a liquid carrier or solvent for the mucoadhesive polymer and absorption enhancer, such as water or aqueous buffer and water miscible cosolvent like glycerine. The resulting solution or suspension will include a solids content within the range from about 0.01 to about 50%, preferably from about 0.1 to about 20%. The solution or suspension will contain a minimum of about 15% by weight liquid carrier or solvent.

Where the composition of the invention is in the form of a semi-solid or gel, it will contain a minimum of about 15% by weight liquid carrier. The semi-solid or gel will include a gelling agent or thickener in an amount within the range from about 0.01 to about 50% by weight, preferably from about 0.1 to about 30% by weight, and a solids content within the range from about 0.1 to about 75% by weight, preferably from about 0.1 to about 50% by weight.

The composition of the invention will not include two liquid phases such as oil-in-water emulsions or water-in-oil emulsions or alcohol containing gels.

The composition of the invention may also be in the form of a solid such as a tablet, bead, beadlet, capsule, powder and the like, although fluid forms as set out above are preferred, all of which include a mixture of the mucoadhesive polymer and absorption enhancer, and not particles containing an absorption enhancer coated with a mucoadhesive polymer.

The composition of the invention will have bioadhesive properties, that is, it will adhere to human or animal mucosa, or adhesive properties will develop on contact with human or animal mucosa. Thus, the composition of the invention, which may be in liquid dosage form, semi-solid dosage form or solid dosage form which may be delivered orally or topically, will adhere to intestinal mucosa, nasal mucosa, buccal mucosa, sublingual mucosa, and vaginal mucosa. The present invention does not include transdermal delivery. The composition of the invention administered in such a manner exhibits reduced mucosal toxicity that is significantly reduced over that typically observed with use of the permeation enhancer alone.

The composition of the invention will include an active pharmaceutical or therapeutic ingredient which may even include pharmaceuticals which have poor oral/intra-oral bioavailability such as peptides, proteins and even poorly permeable small molecules. Examples of pharmaceuticals suitable for use in the composition of the invention include, but are not limited to anti-infectives such as antibiotics and antiviral agents, analgesics and analgesic combinations, anorexics and appetite suppressants, anthelmintics, anesthetics, antiarthritics, antiasthma agents, anticonvulsants, antidepressants, antidiabetic agents, antidiarrheals, antihistamines, anti-inflammatory agents, antimigraine preparations including GLP-1 mimetic peptides, antimotion sickness agents, antinauseants, antineoplastics, antiparkinsonism agents, antipruritics, antipsychotics, antipyretics, antispasmodics, anticholinergics, sympathomimetics, xanthine derivatives, cardiovascular preparations including calcium channel blockers, beta blockers, antiarrhythmics, antihypertensives, diuretics, vasodilators (general, coronary, peripheral and cerebral), erectile dysfunction agents such as selective serotonin reuptake inhibitors and phosphodiesterase inhibitors, central nervous system stimulants, cough and cold preparations, decongestants, diagnostics, hormones, hypnotics, immunosuppressives, muscle relaxants, parasympatholytics, parasympathomimetics, psychostimulants, sedatives, tranquilizers, antioxidants, vitamins, minerals, other nutrients, and herbal extracts or preparations.

Preferred pharmaceuticals for use herein include analogs and derivatives of insulin, glucagon-like peptides (GLP-1 peptides) calcitonin-gene related antagonists, selective serotonin-reuptake inhibitors and growth hormone secretogogues.

Examples of specific pharmaceuticals suitable for use herein include, but are not limited to, acarbose; alendronate; amantadine hydrochloride; azithromycin; calcitonin human; calcitonin salmon; ceftriaxone; cefuroxime axetil; chrionic gonadotropin; cromolyn sodium; daltaperin sodium; danaproid; desmopressin; didanosine; editronate disodium; enoxaprin sodium; epoetin alpha; factor IX; famiciclovir; foscaret sodium; ganciclovir; granulocyte colony stimulating factor; granulocyte-macrophage stimulating factor; growth hormones-recombinant human; growth hormone—Bovine; glucagon; gonadotropin releasing hormone and synthetic analogs thereof; GnRH; gonadorelin; heparin sodium; indinavir sulfate; influenza virus vaccine; interleukin-2; interleukin-3; insulin-human; insulin lispro; insulin porcine; interferon alpha; interferon beta; leuprolide acetate; metformin hydrochloride; nedocromil sodium; neostigmine bromide; neostigmine methyl sulfate; neutontin; octreotide acetate; olpadronate; pamidronate disodium; risedronate; rimantadine hydrochloride; salmeterol; xinafoate; somatostatin; stavudine; ticarcillin; tiludronate; tissue type plasminogen activator; TNFR:Fc; TNK-tPA; tumor necrosis factor; typhoid vaccine live; vancomycin; valaciclovir; vasopressin and vasopressin derivatives; zalcitabine; zanamavir and zidovudine.

Where the composition of the invention is used as a drug delivery vehicle, that is in the form of a liquid dosage form, semi-solid dosage form or solid dosage form, it may include a drug in an amount within the range from about 0.01 to about 90% by weight of the final composition.

The composition of the invention may also serve as a carrier for other active ingredients such as a cosmetic substance, a local or general anesthetic, or analgesic, or an opiate, a vaccine, an antigen, a microorganism, a sterilizing substance, a contraception composition, a protein or peptide such as insulin or calcitonin, or a hormone such as a growth hormone or a seed germination hormone, a steroid, a toxin or a marker substance.

Depending upon the dosage form, whether in liquid or solid form, the composition of the invention will include the mucoadhesive polymer in an amount within the range from about 0.001 to about 75% by weight, preferably from about 0.01 to about 30%, more preferably from about 0.01 to about 10%, and even more preferably from about 0.01 to about 3% by weight of the final composition.

Thus, where the dosage form of the composition of the invention is in the form of a liquid, such as a solution or suspension, the mucoadhesive polymer will be present in an amount within the range from about 0.001 to about 10% and preferably from about 0.01 to about 3% by weight based on the volume of the composition. Where the dosage form of the composition of the invention is in the form of a semi-solid or solid, the mucoadhesive polymer will be present in an amount within the range from about 12 to about 75%, preferably from about 13 to about 50% based on the weight of the composition.

The mucoadhesive polymer employed will be a polycarboxylated polymer having an average molecular weight of at least 10,000 Daltons and up to 4 billion Daltons, preferably at least 500,000 up to 5 million Daltons, and includes polyacrylic acid, cross-linked polyacrylic acid, polyacrylic acid modified by long chain alkyl acrylates, and cross-linked polyacrylic acid modified by long chain alkyl acrylates.

Examples of mucoadhesive polymers suitable for use herein include acrylic acid polymers cross-linked with allyl sucrose, allyl ethers of sucrose, allylpentaerythritol, pentaerythritol or divinyl glycol. Examples of such acrylic acid polymers include polycarbophil polymers available from B.F. Goodrich Specialty Chemicals, Cleveland, Ohio, sold under the trade names Carbopol®, Noveon® and Pemulen®. Preferred mucoadhesive polymers are the pharmaceuticals grades Carbopol 971P, Carbopol 934P (MW 3,000,000) and Carbopol 974P. Most preferred is Carbopol 971P, a lightly cross-linked polyacrylic acid.

Preferred Carboprol cross-linked polyacrylic acid polymers have a molecular weight within the range from 1 million to 5 billion, preferably from about 1.25 to about 4.5 million, a viscosity ranging from 1,000 to 60,000 centipoise, an average particle size ranging from about 0.2 to about 200 μm, preferably from about 1 to about 20 μm, more preferably from about 2 to about 7 μm, a carboxylic acid content ranging from 56 to 68%, a pKa of 6.0±0.5, a solution pH range of pH 2.5 for a concentrated unneutralized solution to pH 7.4 for a neutralized solution, and cross-linking with polyalkenyl ethers or divinyl glycol.

The polyacrylic polymers are preferred because they exhibit multiple functional properties such as:

(1) they increase the residence time at the mucosal surface by adhering to glycocalyx components of the cell surface;

(2) they inhibit enzymes that inactivate peptide and protein drugs (Luesse, H. L. et al., Mucoadhesive polymers in peroral peptide drug delivery. I. Influence of mucoadhesive excipients on the proteolytic activity of intestinal enzymes. Eur. I. Pharm. Sci., 1996, 4:117-128; and

(3) they transiently increase tight junctional permeability (Borchard, G. et al., “The potential of mucoadhesive polymers in enhancing intestinal absorption. III. Effect of chitosan glutamate and carbomer on epithelial tight junctions in vitro.” J. Control. Rel., 1996, 39:131-138.

Polyacrylic polymers enhance permeability of small molecules and macromolecules, and together with an enhancer like LPC have the distinct ability to give an unexpectedly improvement in permeability enhancement.

Depending upon the dosage form, whether in liquid or solid form, the composition of the invention will include the absorption enhancer in an amount within the range from about 0.0001 to about 75% by weight, preferably from about 0.001 to about 30% and more preferably from about 0.01 to 10% by weight of the final composition.

Thus, where the dosage form of the composition of the invention is in the form of a liquid, such as a solution or suspension, the absorption enhancer will be present in an amount within the range from about 0.01 to about 10%, preferably from about 0.05 to about 5% by weight/volume of the final composition. Where the dosage form of the composition of the invention is in the faun of a semi-solid or solid, the absorption enhancer will be present in an amount within the range from about 1 to about 50%, preferably from about 5 to about 30% based on the weight of the final composition.

The absorption enhancer will preferably be a lysophosphatidate, preferably a lysophosphatidylcholine, and more preferably L-α-lysophosphatidylcholine (LPC). However, other lysophosphatidates may be employed such as lysophosphatidylethanolamine, lysophosphatidylglycerol, lysophosphatidylserine, lysophosphatidylinositol, lysophosphatidic acid, cyclic-lysophosphatidic acid and other analogs of lysophosphatidates with multifunctional head group.

The liquid, semi-solid and solid compositions of the invention may be prepared by mixing the individual components, namely the mucoadhesive polymer, absorption enhancer and pharmaceutical with suitable non-toxic pharmaceutically acceptable ingredients known in the art, depending upon the particular dosage form desired, such as disclosed in Remington: The Science and Practice of Pharmacy, 20^{th} edition, Part V (2000).

The liquid formulations of the invention may be in the form of a solution, spray or viscous aqueous gel and may include pharmaceutically acceptable excipients including one or more preservatives, viscosity builders, stabilizing agents, buffers and solubilizing cosolvents as needed for individual active pharmaceutical ingredients.

Preservatives suitable for use in the liquid formulations of the invention include methyl paraben, p-hydroxybenzoic acid esters, chlorbutanol, phenylethyl alcohol, benzathonium chloride and benzalkonium chloride, with methyl paraben being preferred.

Viscosity builders suitable for use in the liquid formulations of the invention include sodium carboxymethyl cellulose, microcrystalline cellulose, polyvinyl pyrrolidone and various gums.

Stabilizing agents such as antioxidants suitable for use in the liquid formulations of the invention include sodium bisulfate and sodium ascorbate, and chelating agents such as EDTA.

Solubilizing cosolvents for a pharmaceutical suitable for use herein will depend upon the pharmaceutical employed and may include polyethylene glycols, propylene glycol, glycerine and ethanol.

Buffers suitable for use in the liquid formulations of the invention include citrate, acetate, phosphate and bicarbonate. pH adjustment can be achieved with hydrochloric acid and sodium hydroxide from about 2.5 to about 8.

Preferred liquid formulations of the invention are set out below.

| | Preferred | More Preferred |
|---|---|---|
| Ingredient | Range (w/v) | Range (w/v) |
| Pharmaceutical | 0.01-30% | 0.1-10% |
| Cosolvent (to solubilize drug) | 0.1-50% | 1-30% |
| Polyacrylic acid polymer | 0.001-10% | 0.01-3% |
| L-α-lysophophatidylcholine | 0.03-5% | 0.05-4% |
| Buffer components | 0.01-2% | 0.1-1.5% |
| Preservative | 0.01-5% | 0.05-0.5% |
| Water | q.s. to 100 ml | q.s. to 100 ml |

A preferred liquid nasal formulation of the invention is set out below.

| Ingredient | Amount | |
|---|---|---|
| Pharmaceutical | 0.01-2 | g |
| PEG-400 | 0.1-15 | ml |
| Polyacrylic acid polymer | 0.5 | g |
| L-α-lysophophatidylcholine | 0.5 | g |
| Methyl paraben | 0.2 | g |
| Sodium chloride | As needed to adjust tonicity | |
| HCl or NaOH | To adjust pH between 2.5-8 | |
| Purified water | q.s. to 100 | ml |

The above solution or liquid, formulations of the invention can be administered as a solution, spray or viscous aqueous gel for nasal delivery. Such solution formulations can also be delivered to the gastrointestinal tract, the buccal/sublingual cavity, the ocular and vaginal mucosa.

The solid formulation of the invention in the form of a tablet or powder to be encapsulated may include pharmaceutically acceptable excipients including one or more fillers or bulking agents, disintegrants, lubricants, binders, stabilizers, and glidants typically used in the formulation of tablets and powders to be encapsulated.

The solid formulation of the invention includes the mucoadhesive polymer and absorption enhancers mixed with the remaining ingredients. The mucoadhesive polymer is not coated on powders, or beads or tablets.

Bulking agents or fillers suitable for use in the solid formulations of the invention include lactose, microcrystalline cellulose, cellulose, hydroxypropyl cellulose, starch, corn starch, modified corn starch, pregelatinized starch, inorganic salts such as calcium carbonate, calcium sulfate, calcium phosphate, and dicalcium phosphate, sugar, dextrose, mannitol, sorbitol or mixtures of two or more thereof.

Disintegrants suitable for use in the solid formulations of the invention include corn starch, potato starch, pre-gelatinized starch, crospovidone, croscarmellose sodium or sodium starch glycollate.

Lubricants suitable for use in tablet formulations of the invention include zinc stearate, magnesium stearate, calcium stearate, talc, carnauba wax, stearic acid, palmitic acid or hydrogenated vegetable oils and fats.

Binders suitable for use in tablet formulations of the invention include corn starch, pregelatinized starch, polyvinyl pyrrolidone (PVP), hydroxypropylmethylcellulose (HPMC), ethyl cellulose, cellulose acetate and the like.

Stabilizers suitable for use in the solid formulations of the invention include antioxidants such as sodium bisulfate and sodium ascorbate, and chelating agents such as EDTA.

Glidants suitable for use in the solid formulations of the invention include colloidal silica dioxide, or talc. Preferred tablet formulations of the invention are set out below.

| | Preferred Range | More Preferred Range |
|---|---|---|
| Ingredient | (% by weight) | (% by weight) |
| Pharmaceutical compound | 1-50% | 6-25% |
| Fillers or Bulking Agents | 15-90% | 25-80% |
| Disintegrants | 0.25-15% | 0.5-5% |
| Lubricants | 0.2-2% | 0.3-0.75% |
| Glidants | 0.01-5% | 0.1-2% |
| Mucoadhesive polymer | 12-75% | 13-50% |
| LPC enhancer | 1-50% | 5-30% |
| Binder | 1-10% | 2-5% |

A preferred tablet formulation for a 300 mg tablet is set out below.

| Ingredient | Amount | |
|---|---|---|
| Pharmaceutical | 1-100 | mg |
| Microcrystalline Cellulose | 50 | mg |
| Hydroxypropyl Cellulose | 45 | mg |
| Croscarmellose Sodium | 4 | mg |
| Magnesium Stearate | 1 | mg |
| Polyacrylic acid polymer | 40 | mg |
| L-α-lysophosphatidylcholine | 40 | mg |

Preferred capsule formulations of the invention are set out below.

| | Preferred Range | More Preferred Range |
|---|---|---|
| Ingredient | (% by weight) | (% by weight) |
| Pharmaceutical | 1-50% | 5-30% |
| Bulking Agent such as | 10-85% | 40-80% |
| Lactose | | |
| Lubricant such as Magnesium | 0.2-2% | 0.3-0.75% |
| Stearate | | |
| Polyacrylic acid polymer | 12-75% | 13-50% |
| L-α-lysophosphatidylcholine | 1-75% | 5-30% |

A preferred capsule formulation of the invention is set out below.

| Ingredient | Amount | |
|---|---|---|
| Pharmaceutical | 1 to 200 | mg |
| Lactose | 200 | mg |
| Polyacrylic acid polymer | 100 | mg |
| Lysophosphatidylcholine | 100 | mg |
| Magnesium stearate | 25 | mg |

The above tablet and capsule formulations can be delivered as described or film-coated to target release in the intestine.

### EXAMPLES

The following Examples represent preferred embodiments of the invention.

### Example 1

A liquid nasal formulation, in accordance with the present invention, having the following composition is prepared as described below.

| Ingredient | Amount | |
|---|---|---|
| Active drug (Desmopressin) | 0.02 | g |
| PEG-400 | 0.1-15 | ml |
| Polyacrylic acid polymer | 0.5 | g |
| L-α-lysophosphatidylcholine (LPC) | 0.5 | g |
| Methyl paraben | 0.2 | g |
| Sodium chloride | As needed to adjust tonicity | |
| HCl or NaOH | To adjust pH between 2.5-8 | |
| Purified water | q.s. to 100 | ml |

Weighed amounts of polyacrylic acid polymer and LPC are added to a portion of water and stirred for about 30 min to completely hydrate the polymer. Active drug ingredient alone or solubilized in a PEG-400 (cosolvent) are added gradually to the stirring solution. All the other inactive ingredients are added with stirring: methyl paraben, NaCl to adjust tonicity, HCl or NaOH to adjust pH. Water is added to the desired target volume.

The above solution formulation can be administered as a solution, spray or viscous aqueous gel for nasal delivery. Likewise, such solution formulations can be delivered to the gastrointestinal tract, the buccal/sublingual cavity, the ocular and vaginal mucosa.

### Example 2

A tablet formulation for a 200 mg tablet, in accordance with the present invention, having the following composition is prepared as described below.

| Ingredient | Amount |
|---|---|
| Active compound (Atenolol) | 25 mg |
| Microcrystalline Cellulose | 45 mg |
| Hydroxypropyl Cellulose | 45 mg |
| Croscarmellose Sodium | 4 mg |
| Magnesium Stearate | 1 mg |
| Polyacrylic acid polymer | 40 mg |
| L-α-lysophosphatidylcholine | 40 mg |

The active pharmaceutical ingredient was blended with microcrystalline cellulose, croscarmellose sodium, hydroxypropyl cellulose (Klucel LF), polyacrylic acid polymer (Carbopol 971P), and LPC in a high shear granulation/mixer. The blend was screened through a 20-mesh screen. Magnesium stearate was added to the final blend in a Turbula® mixer. The lubricated blend was compressed using a single station press into 200 mg tablets of the invention.

### Example 3

A capsule formulation having the following composition was prepared as described below.

| Ingredient | Amount |
|---|---|
| Acyclovir | 100 mg |
| Lactose | 200 mg |
| Polyacrylic acid polymer | 100 mg |
| L-α-Lysophosphatidylcholine | 100 mg |
| Magnesium stearate | 25 mg |

The active pharmaceutical ingredient was blended with lactose, polyacrylic acid polymer (Carbopol 971P), and LPC in a high shear granulation/mixer. The blend was screened through a 20-mesh screen. Magnesium stearate was added to the final blend in a Turbula® mixer. The lubricated blend in the form of a powder was poured into capsules.

The following Examples illustrate the permeation enhancement and cytoprotection of the combination of Carbopol 971P and LPC.

### Example 4

### Permeation Enhancement of DDAVP

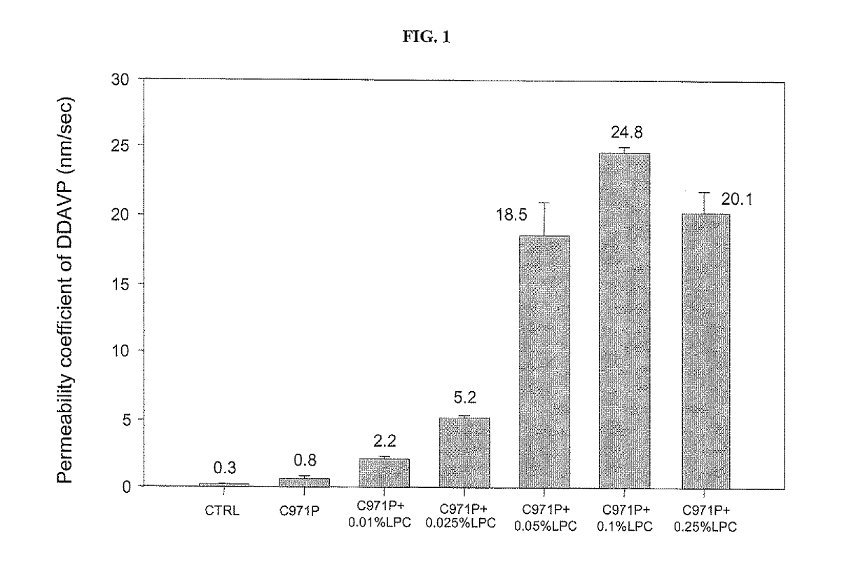

FIG. 1 shows the effect of 0.5% Carbopol 971P and its combination with L-α-lysophosatidylcholine on permeability (Pₐₚₚ) of DDAVP across Calu-3 cells. Pₐₚₚ values are labeled on top of the bars. CTRL was Ca²⁺ and Mg²⁺-free bicarbonated Ringer's solution.

Permeation enhancement of the model peptide DDAVP (MW 1,183) was studied in Calu-3 human bronchial epithelial cells cultured on Transwell permeable filter supports. In confluent cultures, DDAVP dosing solution in buffer or in C971P and/or LPC was added to the apical chamber and the extent of DDAVP transported to the basolateral buffer was estimated by HPLC/UV analysis of the samples at predetermined time intervals. Carbopol 971P (0.5% w/v) by itself produced a 2.7-fold transport enhancement over control (see FIG. 1). Inclusion of LPC further boosted the transport of DDAVP. Pₐₚₚ values were increased dose-dependently with the increase of LPC concentration (0.01-0.25%) for up to 32-fold, compared with 0.5% C971P alone.

### Calu-3 Cell Cultures Employed

Human bronchial epithelial cell line, Calu-3 cells, were cultured in 1:1 Dulbecco's minimum essential medium:F12, supplemented with 10% fetal bovine serum, 2 mM L-glutamine, 50 U/ml of penicillin, 50 μg/ml streptomycin, and 1 μg/ml fungizone in 75 cm² culture flasks. Cells were grown in T-75 flasks to confluence in 7 days. Cells were harvested for seeding onto Transwell filters or passaging into a new flask. Transwell filters (1.13 cm², Corning Costar) were coated with rat tail collagen (30 μg/ml) and cells seeded at a density of 2.5×10⁵ cells/filter, and cultured in a humidified atmosphere of 5% CO₂/95% air at 37° C. After 48 hr, an air-interface was created and the cells were maintained with 0.8 ml of culture medium in the basolateral chambers of Transwells. The air-interface conditions stimulated differentiation of the cell monolayer to form polarized, bioelectrically “tight” epithelial monolayer suitable for transport studies. Day 8-16 monolayers of passage number 22-36 were used in all experiments.

### Example 5

### Acute Cytotoxicity Assay

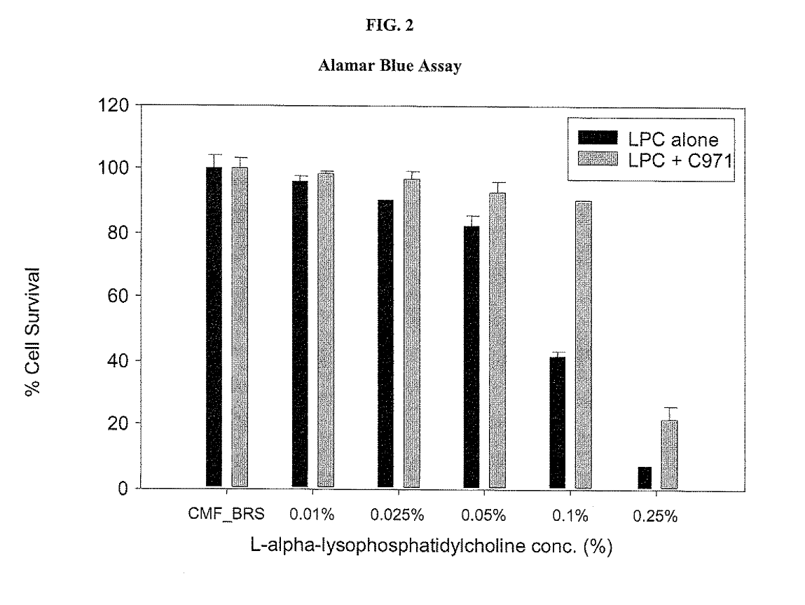

FIG. 2 depicts an Alamar Blue Assay—Black bar indicates the % of the cell survival after 2 hrs treatment of LPC at 0-0.25%; gray bar indicates the combination of LPC with 0.5% of C971P.

### Acute Cytotoxicity Assay

This assay is based on the ability of live cells to enzymatically reduce Alamar blue dye which indicates the relative percentage of cells that survive the exposure to the various treatments. Calu-3 cells exposed to buffer for 2 hr were used as the positive control with 100% cell survival. Cells exposed to LPC alone for 2 hr showed a dose-dependent reduction in survival. When Carbopol 971P was present, cells were protected from the toxicity of LPC (see FIG. 2). About 82% cell survival at 0.05% LPC concentration was increased to 95% with C971P in the formulation. At 0.1% LPC concentration cell survival increased from 41% to >90% in the presence of C971P.

### Example 6

### TEER Measurement (Cell Integrity and Reversibility)

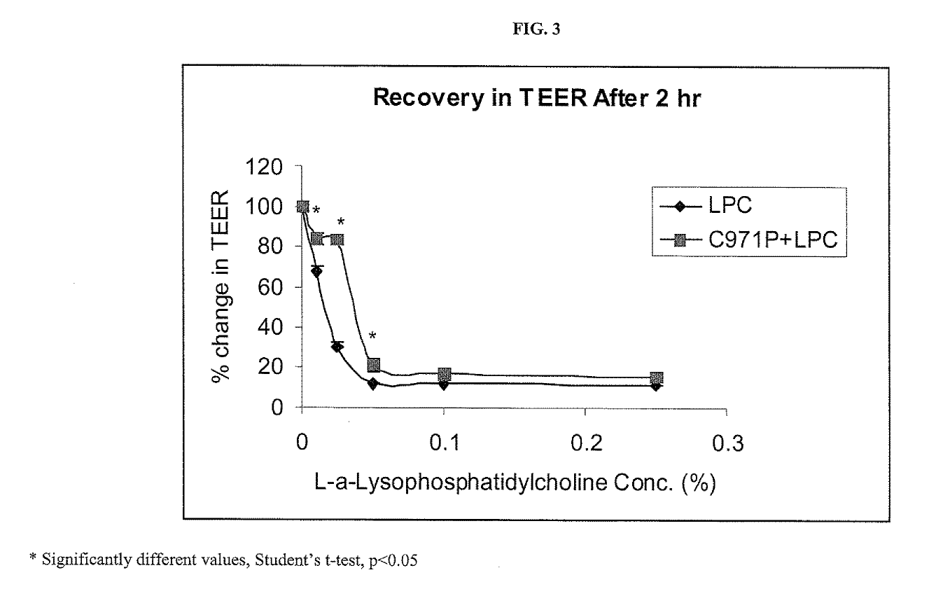

FIG. 3 shows the % of TEER reduction=(TEER_{2 hrs}/TEERᵢₙᵢₜᵢₐₗ)×100. The asterisk (delete scatters with) (*) represent (delete marker present) those samples where TEER recovered to the original level within 12 hrs after washout of the treatment.

Transepithelial electrical resistance (TEER) measures the integrity of the epithelial barrier. When an enhancer is added to the cell monolayer the TEER drops suggesting an increase in epithelial permeability. Return of the TEER values to initial levels indicates recovery to baseline barrier properties. TEER measurements were done in confluent highly resistant Calu-3 cells cultured in Transwell filters with handheld STX-2 electrodes connected to EVOM Voltohmmeter. LPC alone elicited a dramatic drop in TEER. When C971P was present in the formulation, the extent of drop was significantly reduced. After the removal of apical treatment, TEER recovered to the original values. At 0.05% LPC concentration, the cells recovered when C971P was also present, but not with LPC alone.

### Example 7

### Enhancement of DDAVP Absorption in Rat Colon

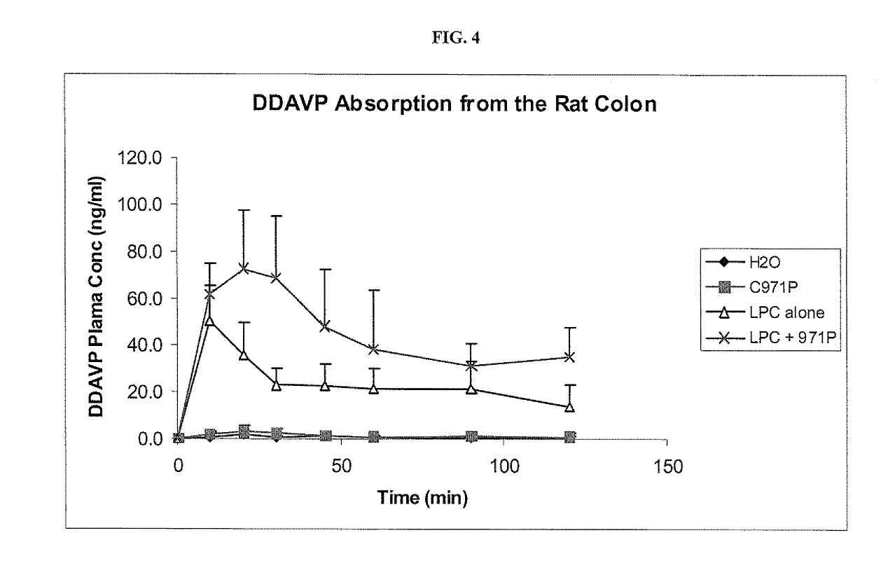

A dosing solution of DDAVP in water, C971P, LPC or C971P/LPC was directly injected into a 10-cm segment of rat colon. Blood was collected over a 2 hr period to assess the extent of systemic absorption from the various formulations. As seen in FIG. 4, at a dose of 1 mg/kg the percent bioavailability for DDAVP was 0.1±0.1% in water, 0.1±0.1% in 0.25% C971P, 2.5±1 in 0.5% LPC, and 4.6±1.7 in C971P/LPC formulation—an enhancement of 46-fold compared to control and >80% compared to LPC alone.

### Example 8

### Rat Colon Reduced Toxicity

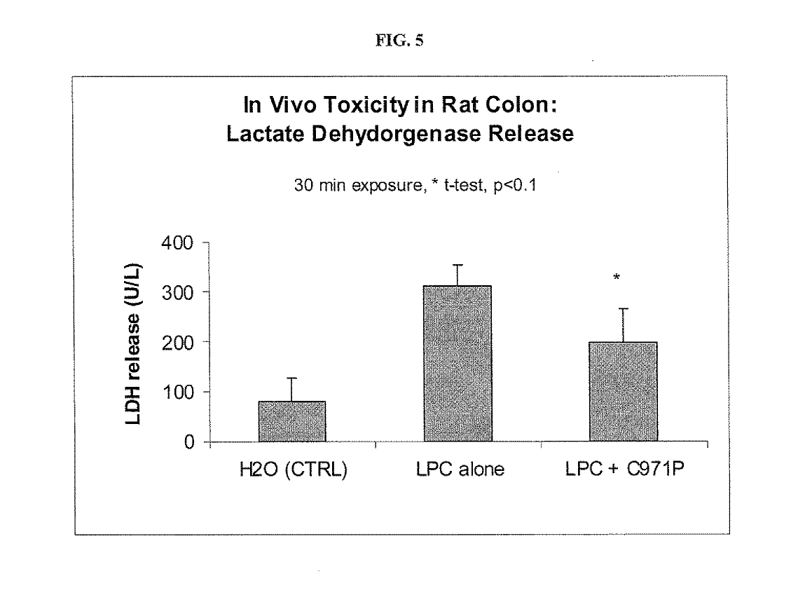

The ability of the C971P/LPC to reduce cytotoxicity in vivo was tested in the rat colon. Water, LPC alone or C971P/LPC solution was introduced into a 10-cm segment of the rat colon and ligated at either end. After 30 min exposure the amount of lactate dehydrogenase, a cytosolic enzyme, released into the lumen was estimated as a measure of mucosal damage from the dosing formulations. As seen in FIG. 5, the C971P/LPC formulation reduced the LDH release by almost 40% compared to LPC alone indicating protection from toxicity of LPC.

### Example 9

### Enhancement of Intra-Nasal DADLE (D-Alanine, D-Leucine-Enkephalin) Absorption in Rabbits

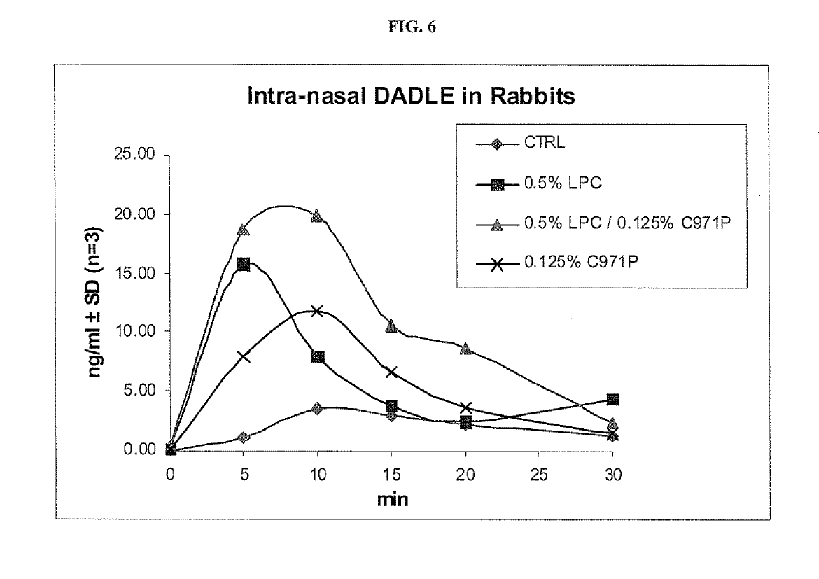

Intra-nasal delivery of the model pentapepdide DADLE in water, C971P, LPC or C971P/LPC formulation was studied in rabbits. As seen in FIG. 6, at a dose of 1 mg/kg delivered via a syringe microsprayer in the rabbit nostril, the exposure in C971P, LPC and the C971P/LPC formulations was 1.9, 2.4 and 3.7-times higher than that of water control, respectively. The C971P/LPC formulation enhanced DADLE absorption by >50% compared to LPC alone.

### Example 10

### Rabbit Nasal Toxicity

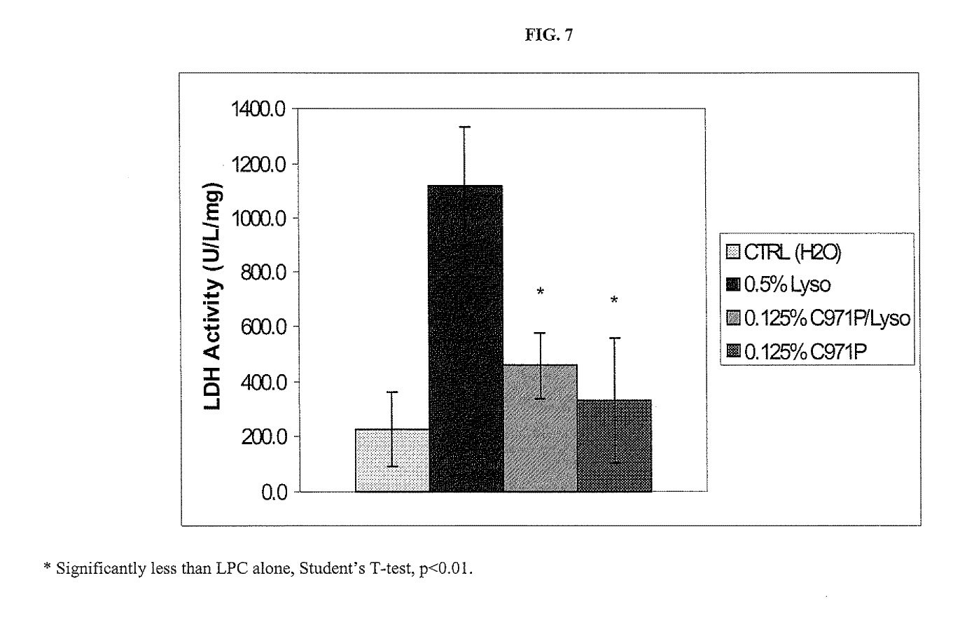

Toxicity of each of the formulations was also tested in the sensitive mucosa of the rabbit nasal cavity. 100 μl of each formulation was administered to each nostril in 3 rabbits and the cavity was lavaged 10-min later. As seen in FIG. 7, the LDH released from the nasal mucosa dosed with the C971P/LPC formulation was 60% lower than LPC alone. The LDH levels from the C971P/LPC formulation were equivalent to that of water.

### Example 11

### Lack of Effect with Phosphatidylcholine (Lecithin) and Other Enhancers

Phosphatidylcholine was disclosed in US2003/0143277 A1 as an enhancer along with bioadhesive polymers such as Carbopol. Phosphatidylcholine failed to show any measurable enhancement or TEER drop. In the Table 1 set out below, it is seen that increasing concentration of phosphatidylcholine did not increase the permeability coefficient of the model peptide DDAVP and neither did it impact TEER 2 hr after exposure. Even when Carbopol was added along with phosphatidylcholine there was no significant enhancement in permeability or drop in TEER.

| Phosphatidylcholine | | | Carbopol 971P + | |
|---|---|---|---|---|
| (Lecithin) Treatment | | | Lecithin | |
| | DDAVP | TEER after | DDAVP | |
| Lecithin Conc. | Permeability | 2 hr (% | Permeability | Drop in |
| (mM) | (nm/sec) | initial) | (nm/sec) | TEER |
| 0 (Control) | 0.4 ± 0.03 | No change | 1.2 ± 0.09 | No change |
| 1 | 0.45 ± 0.03 | No change | 1.1 ± 0.14 | No change |
| 2 | 0.44 ± 0.03 | No change | 1.2 ± 0.08 | No change |
| 5 | 0.44 ± 0.04 | No change | 1.1 ± 0.11 | No change |

Similarly, Carbopol 971P failed to reduce the acute cytotoxicity of the classes of enhancers like fatty acid, e.g. sodium caprate and the chelator, e.g. EDTA in the Alamar Blue cytotoxicity assay in Calu-3 cells. None of these enhancers when combined with Carbopol polymers showed any improvement in permeation enhancement compared to the enhancer alone.

### Example 12

### Lack of Significant Effect of Other Polymers

Other polymers were tested for their ability to enhance peptide permeability compared to Carbopol polymers. Cellulosic polymers like hydroxypropylmethylcellulose (HPMC) and carboxymethyl cellulose (CMC) were found to be 40-50% less effective in enhancing the permeability of the model pepdide, DDAVP compared to Carbopol polymers in Calu-3 cells.

| Polymer Treatment | DDAVP Permeability in |
|---|---|
| (0.25% w/w) | Calu-3 Cells |
| C971P | 28.2 ± 0.7* |
| HPMC | 17.3 ± 1.0 |
| PVA | 12.2 ± 1.2 |
| CMC | 11.4 ± 1.2 |

*Significantly different from other treatments, p < 0.05

Polymer grades that showed similar viscosity at 0.25% w/w were used in this study.

1-10. (canceled)

11. The method as defined in claim 22 wherein the bioadhesive composition comprises a lysophosphatidate and a polyacrylic acid polymer, mixed with each other, where the composition is in the form of a liquid, the polyacrylic acid polymer is present in an amount within the range from about 0.001 to about 10% w/v and the lysophosphatidate is present in an amount within the range from about 0.3 to about 5% w/v, and where the composition is in the form of a semi-solid or solid, the polyacrylic acid polymer is present in an amount within the range from about 12 to about 75% by weight and the lysophosphatidate is present in an amount within the range from about 1 to about 50% by weight, where the mucoadhesive is not a polysaccharide, the enhancer is not an alcohol, and the composition is not an oil-in-water emulsion.

12. (canceled)

13. The method as defined in claim 11, wherein the concentrations of the lysophosphatidate and polyacrylic acid polymer in said composition are effective to provide an enhanced permeability of the pharmaceutical.

14. The method as defined in claim 11, wherein the concentration of the polyacrylic acid polymer is effective to reduce the cytotoxicity of the lysophosphatidate.

15. The method as defined in claim 24, wherein the bioadhesive composition is in the form of an aqueous solution, wherein the polyacrylic acid polymer is present in a concentration of from about 0.01 to about 3% w/v of the mucoadhesive composition, and the lysophosphatidate is present at a concentration of from about 0.01 to about 5% w/v of the mucoadhesive composition.

16. The method as defined in claim 24 wherein the bioadhesive composition is in the form of a solid wherein the polyacrylic acid polymer is present in a concentration of from about 13 to about 50% by weight and the lysophosphatidate is present in a concentration of from about 5 to about 30% by weight.

17. (canceled)

18. The method as defined in claim 16 wherein the lysophosphatidate is α-lysophosphatidylcholine.

19. The method as defined in claim 22 wherein the bioadhesive composition is in the form of a liquid having the following formulation

| Ingredient | Range (w/v) |
|---|---|
| Pharmaceutical | 0.01-30% |
| Cosolvent (to solubilize drug) | 0.1-50% |
| Polyacrylic acid polymer | 0.01-5% |
| L-α-lysophosphatidylcholine | 0.03-5% |
| Buffer components | 0.01-2% |
| Preservative | 0.01-5% |
| Water | q.s. to 100 ml |

20. (canceled)

21. (canceled)

22. A method for improving bioavailability of a pharmaceutical which has poor absorption properties, which comprises delivering said pharmaceutical to the mucosal surfaces of a patient in need of treatment, excluding the mucosa of the eye, together with a bioadhesive composition consisting essentially of a solution of or a mixture of a mucoadhesive polymer and an absorption enhancer, and where the composition is in the form of a liquid, the mucoadhesive polymer is present in an amount within the range from about 0.001 to about 10% w/v and the absorption enhancer is present in an amount within the range from about 0.03 to about 5% w/v, and where the composition is in the form of a semi-solid or solid, the mucoadhesive polymer is present in an amount within the range from about 12 to about 75% by weight and the absorption enhancer is present in an amount within the range from about 1 to about 50% by weight where the said mucoadhesive is free of polysaccharides, and the enhancer is free of alcohols or oils.

23. The method as defined in claim 22 wherein the pharmaceutical, mucoadhesive polymer and absorption enhancer are administered to the mucosal membranes of the gastrointestinal tract, nose and oral cavity, sublingual, buccal, or vagina, but not ocular mucosa or skin.

24. The method as defined in claim 22 wherein the mucoadhesive polymer is a polyacrylic acid polymer and the absorption enhancer is a lysophosphatidylcholine, wherein the concentrations of the polyacrylic acid polymer and the absorption enhancer are effective to provide an enhanced permeability of the pharmaceutical while reducing toxicity of the absorption enhancer.

25. The method as defined in claim 22 wherein the mucoadhesive polymer is Carbopol 971P and the absorption enhancer is L-α-lysophosphatidylcholine.

26. A method for reducing the cytotoxic effect of an absorption enhancer, which comprises administering said absorption enhancer together with a mucoadhesive polymer and a pharmaceutical to a patient in need of treatment whereby permeation of said pharmaceutical into local tissue or systemic circulation is enhanced while expected cytotoxic effect of the absorption enhancer is reduced.

27. The method as defined in claim 26 wherein the mucoadhesive polymer is a polyacrylic acid polymer.

28. The method as defined in claim 26 wherein the mucoadhesive polymer is Carbopol 971P.

29. The method as defined in claim 26 wherein the absorption enhancer is L-α-lyso-phosphatidylcholine and the mucoadhesive polymer is Carbopol 971P polymer.

30. The method as defined in claim 26 wherein the absorption enhancer is L-α-lyso-phosphatidylcholine (LPC) and the mucoadhesive polymer is Carbopol 971P and wherein the LPC is employed in a weight ratio to the Carbopol 971P within the range from about 0.1:1 to about 10:1.

31. The method as defined in claim 30 wherein the pharmaceutical are anti-infectives, antibiotics, and antiviral agents, analgesics and analgesic combinations, anorexics and appetite suppressants, anthelmintics, anesthetics, antiarthritics, antiasthma agents, anticonvulsants, antidepressants, antidiabetic agents, antidiarrheals, antihistamines, anti-inflammatory agents, antimigraine preparations, antimotion sickness agents, antinauseants, antineoplastics, antiparkinsonism agents, antipruritics, antipsychotics, antipyretics, antispasmodics, anticholinergics, sympathomimetics, xanthine derivatives, cardiovascular preparations, calcium channel blockers, beta blockers, antiarrhythmics, antihypertensives, diuretics, vasodilators general, coronary, peripheral and cerebral agents erectile dysfunction agents, central nervous system stimulants, cough and cold preparations, decongestants, diagnostics, hormones, hypnotics, immunosuppressives, muscle relaxants, parasympatholytics, parasympathomimetics, psychostimulants, sedatives, tranquilizers, antioxidants, vitamins, minerals, and herbal extracts or preparations or combinations thereof.
